(19) United States
(12) Patent Application Publication
(10) Pub. No.: US 2024/0307227 A1
(43) Pub. Date: Sep. 19, 2024

(54) METHOD FOR PROVIDING CONTROL DATA FOR AN OPHTHALMOLOGICAL LASER OF A TREATMENT APPARATUS, TREATMENT APPARATUS, COMPUTER PROGRAM AND COMPUTER-READABLE MEDIUM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/603,374

(22) Filed: Mar. 13, 2024

(30) Foreign Application Priority Data
Mar. 15, 2023 (DE) 10 2023 106 466.5

(51) Int. Cl. A61F 9/008

(52) U.S. Cl. CPC A61F 9/00804; A61F 9/00825; A61F 2009/00872

(57) ABSTRACT

The invention relates to a control device (18), to a treatment apparatus (10) and to a method for providing control data for an ophthalmological laser (12) of a treatment apparatus (10). As steps, the method includes ascertaining (S10) an initial correction value for correcting a visual disorder of a cornea (16) from predetermined examination data; ascertaining (S12) epithelial layer parameters from the predetermined examination data and providing an epithelial layer regeneration model, which describes a regeneration of an epithelial layer with the ascertained epithelial layer parameters; determining (S14) an adapted correction value depending on the initial correction value and the epithelial layer regeneration model; and providing (S16) the control data for the ophthalmological laser (12), which includes the adapted correction value.

### FIELD

The invention relates to a method for providing control data for an ophthalmological laser of a treatment apparatus, to a treatment apparatus, to a computer program and to a computer-readable medium.

### BACKGROUND

Treatment apparatuses and methods for controlling eye surgical lasers for correcting an optical visual disorder and/or pathologically or unnaturally altered areas of a cornea of a patient are known in the prior art. Therein, a pulsed laser and a beam focusing device may for example be formed such that laser pulses effect a photodisruption and/or photablation in a focus situated within the organic tissue to remove tissue from the cornea. In order that a transition from the removed tissue to the cornea is not too abrupt, for example too steep, a transition zone is usually provided in edge areas of the tissue to be removed, which provides a flattened transition for the tissue to be removed in the cornea.

Therein, today's systems and methods for example allow generic treatment methods or treatment methods adapted to the respectively treated patient. For example, adapted treatment methods consider topographic information of the cornea of the respective patient or information to imaging errors or information to wavefronts (related to light, which is incident on an eye of the respective patient).

However, it often becomes apparent that an actually achieved correction of a corneal curvature of an eye deviates from a planned correction and/or undesired aberrations are generated by a treatment. Thus, a result of a correction may turn out unsatisfactory.

### SUMMARY

In an aspect, the disclosure provides a correction profile for the treatment of a cornea, by which an improved correction of a cornea is achieved.

The object of the invention is solved by the independent claims. Further advantageous embodiments and modifications of the invention are defined in the dependent claims and/or disclosed in the description as well as the figures.

The invention is based on the idea that regeneration processes of the cornea, in particular regrowth of the epithelial layer, are modeled based on examination data in the treatment planning to thereby obtain an adapted correction profile for achieving the planned correction.

An aspect of the invention relates to a method for providing control data for an ophthalmological laser of a treatment apparatus, wherein the method comprises the following steps performed by a control device. The method includes ascertaining an initial correction value for correcting a visual disorder of a cornea from predetermined examination data, ascertaining epithelial layer parameters from the predetermined examination data and providing an epithelial layer regeneration model, which describes a regeneration of an epithelial layer from the ascertained epithelial layer parameters. Further, the method includes determining an adapted correction value depending on the initial correction value and the epithelial layer regeneration model and providing the control data for the ophthalmological laser, which includes the adapted correction value.

In other words, the initial correction value, which may be ascertained from examination data for an eye of the patient, may be changed by considering the regeneration model of the epithelial layer, to suitably consider a regeneration of the epithelial layer, which may occur after a treatment of the cornea of the eye of the patient. By the method, an adapted correction value may be provided, based on which the control data for the ophthalmological laser may be provided.

The control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

The initial correction value may characterize the correction desired for the patient or the cornea thereof. For example, the initial correction value may be derived from a previously measured surface of the cornea and/or from a previously determined visual disorder. For example, the correction value may indicate a value in diopters, in particular for a spherical and/or cylindrical correction, and the associated correction profile may for example be an ablation profile or an ablation map. Thus, the correction profile or the correction value provides an originally planned correction of the cornea. For example, the visual disorder may have been determined within the scope of a so-called refractometry, for instance at an ophthalmologist. Thus, the initial correction value may be derived from a result of the refractometry or from refractometry data relating to the refractometry. As the initial correction value or as a part of the initial correction value, a desired change of a refractive power of the cornea, a desired change of a curvature of the cornea, a desired size (for example diameter or radius) of an optical zone and/or a desired position of the optical zone related to the cornea are for example determined. Therein, the desired curvature may be indicated in the form of an absolute value or in the form of a relative value. Therein, an absolute value may directly indicate which curvature the cornea is to have in the area of the optical zone after completed treatment, thus after the corneal correction. An absolute value of the curvature may for example be indicated directly by a radius of curvature or indirectly by a focal length or a refractive power. A relative value of the curvature may for example be indicated analogously by a difference of the radius of curvature or by a difference in the focal length or refractive power. Therein, the difference optionally may be respectively related to the state of the cornea before the first corneal correction or after the first corneal correction. Of course, the curvature may also be described by any other suitable physical quantity, whether absolute or relative to the current curvature.

Therein, the epithelial layer parameters may describe a local thickness of the epithelial layer before the treatment among other things, which is ascertained from the examination data of the patient. Further, the epithelial layer parameters may for example include a local regeneration rate and/or a local ablation rate. For example, a local epithelial layer thickness may be ascertained by suitable measurement methods such as e.g. a high-resolution optical coherence tomography (Super OCT). A local regeneration rate and/or a local ablation rate may be ascertained from statistical data or may be derived from long-term observation. Further, the epithelial layer parameters may describe an expectable temporal development or a regeneration capability of the epithelial layer due to the age of the patient, the surface of the treatment, further individual patient parameters and the like among other things.

An epithelial layer regeneration model may be ascertained from the epithelial layer parameters, which may describe a regeneration of an epithelial layer, and may in particular describe a regrowth and/or shift of the epithelial layer in the cornea. In other words, the epithelial layer regeneration model may be a model, which may describe the regeneration of the epithelial layer to be expected within a predetermined period of time after a complete or partial removal or an injury of the epithelial layer, or which may describe a temporal progression of the regeneration of the epithelial layer.

A regeneration of the epithelial layer may be a procedure, which may propagate from the edge of the treatment zone, and it may take some time (usually several weeks to months) until an equilibrium of regrowing epithelial layer and ablated epithelial layer has regenerated after a treatment. For example, this may be taken into account in modeling in that a regeneration of the epithelial layer or of the cornea until the point of time of the equilibrium is simulated by the epithelial layer regeneration model.

The epithelial layer regeneration model may be a mathematical model, which may describe the regeneration procedure by a mathematical formula or relation, or may be a procedure, which may be used to determine an adapted correction value from the initial correction value by considering the regeneration processes of the epithelial layer, which allows an adapted improved correction of the cornea. In the simplest case, a linear model may for example be assumed for regrowth of the epithelial layer. Optionally, the regrowth may be provided as a non-linear model, by which a regeneration rate of the epithelial layer is described depending on an order of magnitude of a local ablation of the epithelial layer.

Using the epithelial layer regeneration model, an adapted correction value may be ascertained based on the initial correction value, which may indicate a refractive power of the cornea or a curvature of the cornea, a size (for example diameter or radius) of an optical zone and/or a position of the optical zone related to the cornea if a regeneration of the epithelial layer is taken into account.

If the epithelial layer regeneration model is not taken into account, it may be expected that a desired correction of a visual disorder by a laser treatment may first be ideal after the treatment, i.e. the visual disorder may initially be corrected completely or as desired. However, without taking the epithelial layer regeneration model into account, a desired correction may degrade in the course of time, since the epithelial layer may regenerate, which may contribute to the refractive power of the eye. This means that the curvature of the cornea may depart from a desired profile by the regeneration processes of the epithelial layer.

However, if the regeneration processes of the epithelial layer are taken into account, i.e. the expected refractive power of the expected epithelial layer after a regeneration is taken into account, it may be expected that the correction of the visual disorder achieved by the treatment may develop from the adapted correction value to the initial correction value by the regeneration of the epithelial layer. For determining the adapted correction value, a profile of the simulated epithelial layer may for example be added to a profile of the planned initial correction, which may be provided by the initial correction value, to compensate for the influence of the epithelial layer regeneration.

Using the techniques presented herein, a better correction of a visual disorder may be achieved, which provides better correction results especially in the long term.

In an embodiment, a method for determining the adapted correction value based on the epithelial layer regeneration model includes ascertaining data of a virtual postoperative cornea, which is expected by the correction using the initial correction value, wherein a regeneration of the epithelial layer is modeled using the epithelial layer regeneration model for the data of the virtual postoperative cornea, ascertaining a virtually achieved correction value from the data of the virtual postoperative cornea and determining the adapted correction value based on the virtually achieved correction value and the initial correction value.

In other words, a virtual cornea may be modeled for determining the adapted correction value, wherein the virtual cornea may represent a cornea after a treatment (i.e. postoperative) with the initial correction value. Therein, the virtual postoperative cornea is modeled using the epithelial layer regeneration model such that it becomes apparent in a virtual model, which virtual correction value is achieved after regeneration of the cornea and to what extent it deviates from the initial correction value. Depending on the virtually achieved correction value arising thereby and the initial correction value, thus, the adapted correction value may be ascertained.

As used herein, “virtual” may mean that a model of the achieved corneal correction is for example simulated using a computer-assisted method to be able to model effects of a planned treatment in this manner.

By ascertaining a virtual postoperative cornea, an adapted correction value may be easier ascertained, whereby a better correction of a visual disorder may be achieved, which provides better correction results especially in the long term. Using the techniques described herein, an adapted correction value may be even more simply ascertained, which additionally even better and more accurately considers the regeneration processes as compared to conventional techniques.

In a further embodiment, it is provided that, in the method, the ascertainment of the data of the virtual postoperative cornea and of the virtual correction value achieved thereto may additionally be repeated for a respectively adapted correction value until the virtually achieved correction value corresponds to the initial correction value.

In other words, the determination of the adapted correction value may be iteratively repeated using the comparison of the virtually achieved correction value to the initial correction value or the respectively adapted correction value to achieve an improved adaptation of the correction value. Further, the above mentioned advantages are also achieved by the invention.

In a further embodiment, it is provided that a difference between the virtually achieved correction value and the initial correction value is determined in the method for determining the adapted correction value, wherein the adaptation of the correction value includes an addition or subtraction of the determined difference with the initial correction value.

In other words, the difference between the virtually achieved correction value and the adapted correction value may be calculated for determining the adapted correction value. This difference or discrepancy may be used in an adaptation of the correction value to obtain an improved adapted correction value.

By calculating the difference, an improved adaptation of the correction value may be achieved. Further, the above mentioned advantages are also achieved by the invention.

In a further embodiment, it is provided that a factor between the virtually achieved correction value and the initial correction value is determined in the method for determining the adapted correction value, wherein the adaptation of the correction value includes a multiplication or division of the determined factor with the initial correction value.

In other words, an adaptation of the correction value with a factor may be performed, which is based on the virtually achieved correction value and the initial correction value. The adaptation using a factor may have advantages to the effect that a faster convergence of the virtually achieved correction value to the initial correction value may be achieved compared to a difference formation. Further, the above mentioned advantages are also achieved by the invention.

In a further embodiment, it is provided that the determination of an adapted correction value is iteratively or repeatedly performed in the method depending on the virtually achieved correction value and the initial correction value.

In particular, the method may be aborted if a difference between the virtually achieved correction value and the initial correction value falls below a predetermined limit value.

By the embodiment, an adapted correction value may additionally be even more simply ascertained, which additionally even more accurately considers the regeneration processes as compared to conventional techniques. If the method according to the above description is aborted, if the difference becomes smaller than the predetermined limit value, thus, a good result may be achieved in shorter time as compared to conventional techniques.

In a further embodiment, it is provided that the epithelial layer regeneration model includes an equation and/or a mathematical procedure in the method.

For example, the epithelial layer regeneration model may use a constant value, which is added up at each location of the remaining residual epithelial layer or by which the thickness of the residual epithelial layer is multiplied (linear regeneration), wherein a thickness of the residual epithelial layer may be ascertained by the epithelial layer parameters of the initial correction value. Further, this value may for example be determined from statistical and/or empirical measurements or may be a non-linear parameter, which is for example determined in the form of a temporal exponential function based on a regeneration rate and an ablation rate of the epithelial layer (e.g. exp(−(regeneration rate)*(ablation of the epithelial layer)*t), wherein t is the time). This is based on the assumption that the epithelial layer more severely or faster regrows in a position, in which it is severely ablated.

By the embodiment, the growth of the epithelial layer may additionally be better modeled to thus be able to ascertain a better adapted correction value, which additionally even more accurately considers the regeneration processes.

In a further embodiment, it is provided that a corneal surface after correction without epithelial layer regeneration model is ascertained in the method for ascertaining the data of the virtual postoperative cornea, wherein a regrowth of the epithelial layer is subsequently calculated for it with the epithelial layer regeneration model.

In other words, for modeling the epithelial layer, a corneal surface may initially be determined, which does not consider regrowth of the epithelial layer, and on which regrowth of the epithelial layer is subsequently modeled by the epithelial layer regeneration model.

By the embodiment, the growth of the epithelial layer may additionally be better modeled to thus be able to ascertain a better adapted correction value, which additionally even more accurately considers the regeneration processes.

In a further embodiment, it is provided that a central optical zone and a transition zone adjoining thereto are provided in the method for correcting the visual disorder, wherein only the transition zone is modified for adapting the correction value.

In other words, the central optical zone may remain unchanged and the adaptation of the correction value may only relate to the transition zone, i.e. the edge zone of the central optical zone, to perform the adaptation.

By the embodiment, the generation of aberrations and/or similar imaging errors may be suppressed, and it may be ensured that a good correction of the visual disorder is not changed.

In a further embodiment, it is provided that a regrowth of the epithelial layer to an original shape of the epithelial layer, which is provided by the epithelial layer parameters, is modeled in the method by the epithelial layer regeneration model.

In other words, e.g. the local thickness of the epithelial layer may be used to model the regrowth of the epithelial layer and/or to ascertain the virtually achieved correction value from the initial correction value. This is based on the assumption that the regrowing epithelial layer may achieve a postoperative local thickness corresponding to the previous local thickness due to regeneration and ablation processes.

By modeling the epithelial layer in this manner, the local thickness of the epithelial layer may additionally be easily modeled in simple manner. Thereby, an improved adaptation of the correction value may be simply achieved.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus. Further, the treatment apparatus and/or the laser may subsequently be controlled with the control data for correcting the cornea.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect relates to a control device, which is formed to perform the steps of at least one embodiment of one or both of the previously described methods. Thereto, the control device may comprise a computing unit for electronic data processing such as, for example, a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect relates to a treatment apparatus with at least one eye surgical or ophthalmological laser and a control device, which is formed to perform the steps of at least one embodiment of one or both of the previously described methods. The respective laser may be formed to at least partially separate a predefined corneal volume with predefined interfaces of a human or animal eye by optical breakthrough, in particular at least partially separate it by photodisruption and/or to ablate corneal layers by (photo)ablation and/or to cause a laser-induced refractive index change in the cornea and/or the eye lens.

A further aspect relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect relates to a computer-readable medium (storage medium), on which the above mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

### BRIEF DESCRIPTION OF THE DRA WINGS

In the following, additional features and advantages of the invention are described in the form of advantageous embodiments based on the figure(s). The features or feature combinations of the embodiments described in the following may be present in any combination with each other and/or the features of the embodiments. This means, the features of the embodiments may supplement and/or replace the features of the embodiments and vice versa. Thus, embodiments are also to be regarded as encompassed and disclosed by the invention, which are not explicitly shown or explained in the figures, but arise from and may be generated by separated feature combinations from the embodiments and/or embodiments. Thus, embodiments are also to be regarded as disclosed, which do not comprise all of the features of an originally formulated claim or extend beyond or deviate from the feature combinations set forth in the relations of the claims. To the embodiments, there shows:

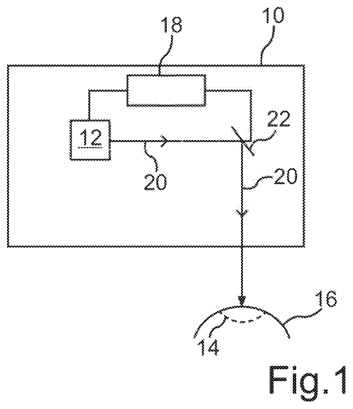

FIG. 1 depicts a schematic representation of a treatment apparatus according to an exemplary embodiment;

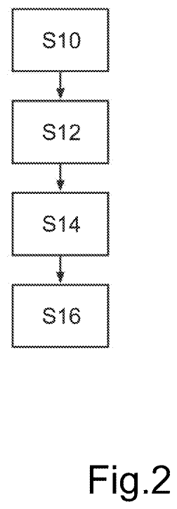

FIG. 2 depicts a flowchart for a method for providing control data according to an exemplary embodiment.

In the figures, identical or functionally identical elements are provided with the same reference characters.

### DETAILED DESCRIPTION

FIG. 1 shows a schematic representation of a treatment apparatus 10 with an eye surgical laser 12 for removing a tissue from a human or animal cornea 16 by photodisruption and/or photoablation. For example, the tissue may be provided by a correction profile 14, by which a volume body may be separated or ablated from the cornea 16 with the eye surgical laser 12 for correcting a visual disorder. This means, a geometry of the tissue to be removed may be provided by the correction profile 14, wherein the correction profile 14 may be preset to a control device 18, in particular in the form of control data, such that the laser 12 emits pulsed laser pulses in a pattern predefined by the control data into the cornea 16 of the eye to remove the tissue. In particular, the correction profile 14 may be provided for achieving at least one predetermined correction value, for example for achieving a predetermined spherical and/or cylindrical correction value. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22, such as for example a rotation scanner, to remove the tissue. The beam deflection device 22 may also be controlled by the control device 18.

The illustrated laser 12 optionally may be a photodisruptive and/or photoablative laser, which is formed to emit laser pulses in a wavelength range between 300 nanometers and 1400 nanometers, optionally between 700 nanometers and 1200 nanometers, at a respective pulse duration between 1 femtosecond and 1 nanosecond, optionally between 10 femtoseconds and 10 picoseconds, and a repetition frequency of greater than 10 kilohertz, optionally between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

After generating the correction profile 14 in the cornea 16, it may occur that it is determined in a follow-up examination that an actually achieved correction deviates from an originally planned correction. This may be ascribed to a regeneration process of an epithelial layer of the cornea 16, which may regrow, and thus more residual tissue is present than planned by the treatment. In order to consider this effect, the method shown in FIG. 2 may be performed.

In FIG. 2, a method for providing control data for the ophthalmological laser 12 of the treatment apparatus 10 is illustrated, wherein the steps may be performed by the control device 18 of the treatment apparatus 10 or the external control device.

In a step S10, an initial correction value or an initial correction profile 14 for correcting a visual disorder of the cornea 16 may be ascertained from predetermined examination data. The correction value may for example indicate a value in diopters, in particular for a spherical and/or cylindrical correction, and the associated correction profile 14 may for example be an ablation profile or an ablation map. Thus, the correction profile 14 provides an originally planned correction of the cornea 16. The visual disorder may for example have been determined within the scope of a so-called refractometry, for instance at an ophthalmologist. For example, a desired change of a refractive power of the cornea, a desired change of a curvature of the cornea, a desired size (for example diameter or radius) of an optical zone and/or a desired position of the optical zone related to the cornea are for example determined as the initial correction value or as a part of the initial correction value. Therein, a central optical zone and a transition zone adjoining thereto may be provided for correcting the visual disorder, wherein only the transition zone may be modified for adapting the correction value.

In a step S12, epithelial layer parameters may be ascertained from the predetermined examination data and an epithelial layer regeneration model may be provided by the epithelial layer parameters, by which a regeneration of the epithelial layer, which has the ascertained epithelial layer parameters, may be described.

Therein, the epithelial layer parameters may describe a local thickness of the epithelial layer before the treatment among other things, which is ascertained from the examination data of the patient. Further, the epithelial layer parameters may for example include a local regeneration rate and/or a local ablation rate. Hereto, the examination data may for example include predetermined data of an optical coherence tomography of the patient. The epithelial layer regeneration model may include an equation and/or a mathematical procedure. For example, a local epithelial layer thickness may be ascertained by suitable measurement methods such as e.g. a high-resolution optical coherence tomography (Super OCT). Further, the epithelial layer parameters may describe an expectable temporal development or a regeneration capability of the epithelial layer due to the age of the patient, the surface of the treatment, further individual patient parameters and the like among other things.

Further, the epithelial layer regeneration model may be a mathematical model, which may describe the regeneration procedure by a mathematical formula or relation. A local regeneration rate and/or a local ablation rate may be ascertained from statistical data or may be derived from long-term observation. Optionally, the epithelial layer regeneration model may be based on a regeneration rate and an ablation rate of the epithelial layer, which may in particular be described by exp(−(regeneration rate)*(ablation of the epithelial layer)*t), wherein t is the time. In the simplest case, a linear model for regrowth of the epithelial layer may be assumed as the epithelial layer regeneration model. For example, the epithelial layer regeneration model may use a constant value, which is added up at each location of the remaining residual epithelial layer or by which the thickness of the residual epithelial layer is multiplied (linear regeneration). Optionally, the regrowth may be provided as a non-linear model, by which a regeneration rate of the epithelial layer is described depending on an order of magnitude of a local ablation of the epithelial layer. Regrowth of the epithelial layer to an original shape of the epithelial layer, which is provided by the epithelial layer parameters, may be modeled by the epithelial layer regeneration model.

In a step S14, an adapted correction value may be determined depending on the initial correction value and the epithelial layer regeneration model. By the epithelial layer regeneration model, an adapted correction value may be ascertained based on the initial correction value, which may indicate a refractive power of the cornea or a curvature of the cornea, a size (for example diameter or radius) of an optical zone and/or a position of the optical zone related to the cornea if a regeneration of the epithelial layer is taken into account.

Further, data of a virtual postoperative cornea, which is expected by the correction by the initial correction value, may be ascertained for determining the adapted correction value depending on the epithelial layer regeneration model, wherein a regeneration of the epithelial layer is modeled by the epithelial layer regeneration model for the data of the virtual postoperative cornea. For ascertaining the data of the virtual postoperative cornea, a corneal surface after correction without epithelial layer regeneration model may be ascertained, wherein regrowth of the epithelial layer may be subsequently calculated for it with the epithelial layer regeneration model.

From the data of the virtual postoperative cornea, a virtually achieved correction value may be ascertained and an adapted correction value may be determined depending on the virtually achieved correction value and the initial correction value.

For a respectively adapted correction value, ascertaining the data of the virtual postoperative cornea and of the virtual correction value achieved thereto optionally may be repeated until the virtually achieved correction value corresponds to the initial correction value. In particular, the method may be aborted if a difference between the virtually achieved correction value and the initial correction value falls below a predetermined limit value.

For determining the adapted correction value, a difference between the virtually achieved correction value and the initial correction value may for example be determined, wherein the adaptation of the correction value may include an addition or subtraction of the determined difference with the initial correction value. For determining the adapted correction value, a factor between the virtually achieved correction value and the initial correction value optionally may be determined, wherein the adaptation of the correction value may include a multiplication or a division of the determined factor with the initial correction value.

Further, the determination of an adapted correction value may be iteratively performed depending on the virtually achieved correction value and the initial correction value.

In a step S16, control data may be provided for the ophthalmological laser, which includes the adapted correction value. This means that the adapted correction profile may be converted into global coordinates for calculating the positioning and laser pulse sequence.

For example, this mathematical procedure may also be used for the correction of higher order aberrations. Furthermore, this procedure may also be converted such that estimations of a postoperative corneal surface, for example for photodisruptive methods, may be used instead of an ablation volume.

Overall, the examples show how a method for providing control data for an ophthalmological laser of a treatment apparatus, a treatment apparatus, a computer program and a computer-readable medium may be provided.

1. A method for providing control data for an ophthalmological laser of a treatment apparatus, wherein the method comprises the following steps performed by a control device:
ascertaining an initial correction value for correcting a visual disorder of a cornea from predetermined examination data;
ascertaining epithelial layer parameters from the predetermined examination data and providing an epithelial layer regeneration model, which describes a regeneration of an epithelial layer with the ascertained epithelial layer parameters;
determining an adapted correction value depending on the initial correction value and the epithelial layer regeneration model; and
providing the control data for the ophthalmological laser, which includes the adapted correction value.

2. The method according to claim 1, wherein for determining the adapted correction value depending on the epithelial layer regeneration model, the following steps are performed:
ascertaining data of a virtual postoperative cornea, which is expected by the correction by the initial correction value, wherein the regeneration of the epithelial layer is modeled by the epithelial layer regeneration model for the data of the virtual postoperative cornea;
ascertaining a virtually achieved correction value from the data of the virtual postoperative cornea; and
determining the adapted correction value depending on the virtually achieved correction value and the initial correction value.

3. The method according to claim 2, wherein ascertaining the data of the virtual postoperative cornea and of the virtual correction value achieved thereto is repeated for a respectively adapted correction value until the virtually achieved correction value corresponds to the initial correction value.

4. The method according to claim 2, wherein for determining the adapted correction value, a difference between the virtually achieved correction value and the initial correction value is determined, wherein the adaptation of the correction value includes an addition or subtraction of the determined difference with the initial correction value.

5. The method according to claim 2, wherein for determining the adapted correction value, a factor between the virtually achieved correction value and the initial correction value is determined, wherein the adaptation of the correction value includes a multiplication or division of the determined factor with the initial correction value.

6. The method according to claim 2, wherein the determination of the adapted correction value is iteratively performed depending on the virtually achieved correction value and the initial correction value.

7. The method according to claim 2, wherein for ascertaining the data of the virtual postoperative cornea, a corneal surface after correction without an epithelial layer regeneration model is ascertained, wherein regrowth of the epithelial layer is subsequently calculated for it with the epithelial layer regeneration model.

8. The method according to claim 1, wherein the epithelial layer regeneration model includes an equation and/or a mathematical procedure.

9. The method according to claim 1, wherein a central optical zone and a transition zone adjoining thereto are provided for correcting the visual disorder, wherein only the transition zone is modified for adapting the correction value.

10. The method according to claim 1, wherein a regrowth of the epithelial layer to an original shape of the epithelial layer, which is provided by the epithelial layer parameters, is modeled by the epithelial layer regeneration model.

11. A method for controlling a treatment apparatus, wherein the method includes:
the method according to claim 1, and
transferring the provided control data to a respective eye surgical laser of the treatment apparatus.

12. A control device, which is configured to perform a respective method according to claim 1.

13. A treatment apparatus with at least one ophthalmological laser for separation of a corneal volume of a human or animal eye by optical breakthrough, in particular by photodisruption and/or photoablation, and at least one control device according to claim 12.

14. (canceled)

15. A computer-readable medium, on which a computer program is stored, the computer program including commands, which cause a treatment apparatus to execute the method according to claim 1.
